(19) United States
(12) Patent Application Publication
(10) Pub. No.: US 2015/0301049 A1
(43) Pub. Date: Oct. 22, 2015

(54) METHOD FOR DIAGNOSING AND TREATING KIDNEY INJURY OR DISEASE

(71) Applicant: THE BRIGHAM AND WOMEN'S HOSPITAL, INC., Boston, MA (US)

(72) Inventor: Jing Zhou, Boston, MA (US)

(73) Assignee: THE BRIGHAM AND WOMEN'S HOSPITAL, INC., Boston, MA (US)

(21) Appl. No.: 14/647,261

(22) PCT Filed: Dec. 3, 2013
(86) PCT No.: PCT/US13/72777

§ 371 Date: May 26, 2015

Related U.S. Application Data
(60) Provisional application No. 61/732,663, filed on Dec. 3, 2012.

(51) Int. Cl. G01N 33/573; A61K 38/17; C12Q 1/68

(52) U.S. Cl. CPC G01N 33/573; C12Q 1/6883; A61K 38/1709; C12Q 2600/112; C12Q 2600/158; G01N 2800/7019; G01N 2333/4703

(57) ABSTRACT

The protein kinase C and casein kinase 2 substrate in neurons (Pacsin) is a subfamily of membrane-binding proteins, participates in vesicle trafficking and cytoskeleton organization. Pacsin 2 expression is significantly upregulated during the repair phase of post ischemia-reperfusion injured (IRI) kidneys, especially on the apical brush border of proximal tubules, which experienced massive damage. Described herein are methods, kits and systems for the diagnosis, selection and treatment of kidney injury and kidney diseases.

### CROSS REFERENCE TO RELATED APPLICATIONS

This application claims benefit under 35 U.S.C. §119(e) of U.S. Provisional Application No. 61/732,663 filed Dec. 3, 2012, the contents of which are incorporated herein by reference in its entirety.

### GOVERNMENT SUPPORT

This invention was made with government support under Grant Nos. DK40703, DK51050 and DK074030 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Dec. 3, 2013, is named 043214-076271-PCT_SL.txt and is 4,644 bytes in size.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Pacsin 2 is a member of the Pacsin (protein kinase C and casein kinase 2 substrate in neurons) protein family. To date, there are three known members in the Pacsin protein family. Pacsin 1 localizes specifically to neurons, Pacsin 3 is mainly detected in the lung and the muscle, whereas Pacsin 2 has a ubiquitous distribution and has a high expression in the kidney⁽¹⁾. Pacsin family proteins are characterized by a highly conserved amino terminal Bin-Amphiphysin-Rvs (F-Bar) domain. Structural studies revealed that the F-Bar domain is required for dimerization to form a crescent or S-shaped structure and is essential for sensing, stabilizing and inducing changes in membrane topology such as during endocytosis^{(2),(3),(4),(5)}. Pacsins localize to sites of high actin turnover, such as filopodia tips ogy 3 (SH3)-domain with the neural Wiskott-Aldrich syndrome protein (N-Wasp⁽¹⁾, which is a potent activator of the Arp2/3 complex that functions in actin filament nucleation^{(6),(7),(8)}, the rate limiting step for actin filament polymerization⁽⁹⁾. The Pacsin 1-N-WASP— Arp2/3 complex-dependent actin nucleation is essential for proper neuromorphogenesis; and similar to the loss of N-WASP and Arp3, the loss of Pacsin 1 results in improper axon development⁽¹⁰⁾. Interestingly, N-Wasp deficiency causes tubulogenesis defects in madin darty canine kidney (MDCK) cells, and reduces the branching and tubule extension in 3D cultures⁽¹¹⁾. Previous studies on Pacsin 2 mostly used non-epithelial cell models^{(1),(12),(13),(14),(15),(16)}. Its expression in kidney development and repair after injury is not known. Whether Pacsin 2 plays a role in tubulogenesis has not been investigated previously.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of diagnosing kidney injury or a kidney disease, optionally selecting a kidney therapy, and optionally administering the kidney therapy, comprising: assaying a biological sample obtained from a subject to determine a Pacsin 2 expression level; comparing the Pacsin 2 expression level to a reference level; and diagnosing the kidney injury or a kidney disease if the Pacsin 2 expression level is higher than the reference level.

In various embodiments, the method can further comprise selecting a kidney therapy if the kidney injury or the kidney disease is diagnosed. In various embodiments, the method can further comprise administering the kidney therapy if the kidney injury or kidney disease is diagnosed.

In various embodiments, the biological sample used in the method can comprise blood or urine. In other embodiments, biological sample used in the method can comprise a kidney cell, kidney tissue, a brush border of a tubule of a kidney, an apical brush border of a proximal tubule of a kidney, or combinations thereof.

In various embodiments, the kidney injury or kidney disease diagnosed, and optionally treated can be post ischemia-reperfusion injury. In other embodiments, the kidney disease can be polycystic kidney disease.

In various embodiments, selecting the kidney therapy can comprise selecting a Pacsin 2 product, a Pacsin 2 product mimetic and combinations thereof. In various embodiments, administering the kidney therapy can comprise administering a therapy from the group consisting of a Pacsin 2 agonist, a Pacsin 2 product, a Pacsin 2 product mimetic and combinations thereof. In various embodiments, the Pacsin 2 product can comprise a Pacsin 2 protein or an active fragment thereof.

Various embodiments of the present invention provide for a system for diagnosing a kidney injury or kidney disease in a subject in need thereof, comprising: a sample analyzer configured to produce a signal for a Pacsin 2 in a biological sample of the subject; and a computer sub-system programmed to calculate, based on the Pacsin 2 level whether the signal is higher than a reference level.

Various embodiments of the present invention provide for a computer program product embodied in a non-transitory computer readable medium that, when executing on a computer, performs steps comprising: detecting a Pacsin 2 expression level in a biological sample from a subject in need of a diagnosis regarding a kidney injury or kidney disease; and comparing the Pacsin 2 expression level to a reference level.

Various embodiments of the present invention provide for a kit for diagnosing a kidney injury or kidney disease in a subject in need thereof, comprising: one or more probes comprising a combination of detectably labeled probes for the detection of Pacsin 2. In various embodiments, the kit can further comprise a computer program product embodied in a non-transitory computer readable medium that, when executing on a computer, performs steps comprising: detecting the Pacsin 2 expression level in a biological sample from a subject in need of a diagnosis regarding a kidney injury or kidney disease; and comparing the Pacsin 2 expression level to a reference level.

Various embodiments of the present invention provide for a method of selecting a kidney therapy, and optionally administering the kidney therapy, comprising: assaying a biological sample obtained from a subject to determine a Pacsin 2 expression level; comparing the Pacsin 2 expression level to a reference level; diagnosing a kidney injury or kidney disease if the Pacsin 2 expression level is higher than the reference level; and selecting a kidney therapy if the kidney injury or kidney disease is diagnosed.

In various embodiments, the method can further comprise administering the kidney therapy if the kidney injury or kidney disease is diagnosed.

In various embodiments, the biological sample used in the method can comprise blood or urine. In other embodiments, the biological sample used in the method can comprise a kidney cell, kidney tissue, a brush border of a tubule of a kidney, an apical brush border of a proximal tubule of a kidney, or combinations thereof.

In various embodiments, the kidney injury or kidney disease wherein a therapy is selected for and optionally administered to treat can be post ischemia-reperfusion injury. In other embodiments, the kidney disease can be polycystic kidney disease.

In various embodiments, selecting the kidney therapy can comprise selecting a therapy from the group consisting of a Pacsin 2 agonist, a Pacsin 2 product, a Pacsin 2 product mimetic and combinations thereof.

In various embodiments, administering the kidney therapy can comprise administering a therapy from the group consisting of a Pacsin 2 agonist, a Pacsin 2 product, a Pacsin 2 product mimetic and combinations thereof. In various embodiments, the Pacsin 2 product can comprise a Pacsin 2 protein or an active fragment thereof.

Various embodiments of the present invention provide for a method of treating a kidney injury or kidney disease, comprising: administering a therapy from the group consisting of a Pacsin 2 agonist, a Pacsin 2 product, a Pacsin 2 product mimetic and combinations thereof to a subject in need thereof. In various embodiments, the Pacsin 2 product can comprise a Pacsin 2 protein or an active fragment thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1B show that MTT assay (A) and FACS (B) analyses show the cell proliferation rate and the cell cycle profile are similar between stable Pacsin 2 knockdown and control mIMCD3 cell lines.

FIG. 1C shows a graph demonstrating the length of the primary cilium. Control cells had an average cilium length of 1.45 uM (n=658) as compared with 1.90 uM (n=844) in knockdown cells. Error bars represent±standard deviation. (n=3). The significance was calculated by Student's T-test.

FIG. 2 shows the quantification of structures formed in 3D tubulogenesis assified into three categories (tubule, cell cluster, and cell chain). The percentage of each type of the structures was calculated by dividing by total number of structures formed. Cell structures were counted from four individual experiments. More than 160 structures were counted for each cell type. Error bars represent±standard deviation.

FIG. 3 shows that the quantification of cell invasion. The number of cells traveled to the basal surface in 12 separated fields were counted and divided by the starting number of cells. The control mIMCD3 cells were used for reference and set at 100%. Depleting Pacsin 2 impaired mIMCD3 cells travel through a permeable membrane. Error bars represent±standard deviation. (n=3). The significance was calculated by Student's T-test. P<0.01.

FIG. 4 shows that protein levels of Pacsin 2 were normalized to those of total Erk1/2 in each kidney; and the non-ischemic control kidneys were used as reference and set at 100%. Error bars represent±standard deviation. (n=3). The significance was calculated by Student's T-test. P<0.05.

FIG. 5 depicts the Pacsin 2 protein levels were normalized to β-Actin in each kidney; and the newborn kidneys were used for the reference and set at 100%. Error bars represent±standard deviation. (n=3). The significance was calculated by Student's T-test. P<0.05.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described.

Described herein, we reported that Pacsin 2 is differentially expressed in different nephron segments. Its expression in specified nephron segments coincides with kidney development and kidney repair post ischemia-reperfusion injury (IRI). Pacsin 2 deficient murine inner medullary collecting duct 3 (mIMCD3) cells exhibit disordered tubule formation, resulting in multi-lumen structures in 3-dimensional (3D) collagen gels. In this study, we also show that Pacsin 2 localizes on the primary cilia, and modulates cilium length in kidney epithelial cells. The primary cilium defects cause a number of human genetic diseases, including polycystic kidney diseases (PKD), and Bardet-Biedle syndrome (BBS), collectively termed ciliopathy⁽¹⁷⁾. Taken together, our results suggest that Pacsin 2 contributes to kidney development and repair.

We report a detailed characterization of Pacsin 2 localization in cultured kidney cells and in vivo. We identified Pacsin 2 as a new player in controlling the formation of kidney tubules and the length of primary cilia in kidney epithelial cells. Moreover, we report a striking upregulation of Pacsin 2 in kidneys after ischemia-perfusion injury.

During kidney development, mesenchymal cells induce growth and repeated branching of the ureteric bud. Reciprocally, the tips of the branching ureteric bud, where Pacsin 2 is expressed, induce the surrounding mesenchymal cells to condense into renal vesicles, which ultimately differentiate into various segments of the nephron^{(22),(23),(24)}. F-actin is known to be expressed apically in the tips of ureteric buds and is essential for branching morphogenesis in the developing kidney⁽²⁵⁾. The apical localization of Pacsin 2 in the ureteric buds is consistent with the notion that Pacsin 2 localizes to high actin turnover sites, and modulates actin cytoskeleton reorganization. The data suggest that Pacsin 2 may participate in the regulation of branching morphogenesis in the developing kidney through modulation of the actin cytoskeleton.

Pacsin 2 expression appears to be downregulated in proximal tubules after postnatal kidney development, whereas its apical localization in collecting ducts remains. In embryonic day 15.5 and newborn kidneys, Pacsin 2 is present in the apical membrane in both weeks of age or older, when postnatal development is complete, Pacsin 2 expression decreases in proximal tubules and becomes more cytoplasmically localized. This is in contrast to a retained apical membrane expression of Pacsin 2 in collecting tubules. These data suggest that Pacsin 2 may play different roles in different nephron segments. Pacsin 2 may be more important in proximal tubules during early postnatal kidney development; and is needed for the maintenance of the structure and function of collecting system in developed kidneys. The presence of Pacsin 2 on both the apical surfaces of parietal epithelial cells of the Bowman's capsule and podocytes of the glomerular tufts of the adult kidney suggests that it might contribute to glomerular filtration in addition to its regulation of tubular structure and/or function.

Depleting Pacsin 2 in mIMCD3 cells leads to remarkable defects in 3D tubulogenesis assays. Pacsin 2 deficient cells form cell cords without an open lumen or cell clusters with irregular multiple lumens, in contrast to the control cells that form tubules with a continuous lumen and elaborate branching. Tubulogenesis requires proper control of cell-cell adhesion, cell polarity and cell invasion^{(11, 19)}. Pacsin 2 depleted cells retain primary cilia on the luminal side and normal localization of markers for cell-cell adhesion and cell polarity when cultured either on permeable filters or in 3D collagen gels. Therefore, the defect in tubule formation in Pacsin 2-depleted cells likely results from defective cell invasion, similar to the reduced branching and reduced tubule extension observed in 3D culture of N-WASP deficient MDCK cells⁽¹¹⁾. Corroboratively, transwell cell invasion assays revealed a remarkable defect in Pacsin 2 depleted mIMCD3 cells. Pacsin 2 knockdown causes defects in cell invasion but not apical-basal polarity. Equal number of Pacsin 2 knockdown and control mIMCD3 cells was seeded into the apical chamber of collagen I coated transwell filer (8 μm), and allowed to travel to the underside of the filters for 16 hours. HGF (20 ng/ml) was added to the lower chamber. Migrated cells were stained by Giemsa, and photographed with an inverted phase contrast microscope. Pores within the surface of the filter were seen. (Data not shown.) Quantification of cell invasion was performed. The number of cells traveled to the basal surface in 12 separated fields were counted and divided by the starting number of cells. The control mIMCD3 cells were used for reference and set at 100%. Depleting Pacsin 2 impaired mIMCD3 cells travel through a permeable membrane. The significance was calculated by Student's T-test. P<0.01. (Data not shown.) In addition, Pacsin 2 has recently been shown to bind to Rac1⁽²⁶⁾ as well as cyclin D1⁽²⁷⁾ through its SH3 domain and negatively regulate cell migration in fibroblast and cancer cells.

Primary cilia may function as mechano- and/or chemosensors critical for cted Pacsin 2 on the primary cilia and found that Pacsin 2 knockdown cells possess longer (˜31%) primary cilia than control cells. Given the requirement of Pacsin 2 for proper activity of the Arp2/3 complex^{(1),(6),(7),(8)} it is likely that the defects in the actin cytoskeleton in Pacsin 2 knockdown cells are responsible for the increased length of primary cilia. This is supported by a recent report which shows that Arp3 depletion or inhibition of actin polymerization results in longer cilia by stabilizing a previously unknown pericentrosomal preciliary compartment, a compact vesiculotubular structure that stores ciliary protein during the early phase of ciliogenesis⁽²⁸⁾. Since the length of primary cilia is tightly controlled and its alteration contributes to the onset and progression of many ciliopathies including polycystic kidney disease (PKD), the altered length of primary cilia in Pacsin 2 knockdown cells supports that Pacsin 2-N-Wasp-dependent actin nucleation may control the formation and maintenance of the normal kidney tubular structure via modulating the length of the primary cilium, in addition to its key role in cell invasion during tubulogenesis.

Ischemia-reperfusion injury is a major cause of acute renal failure in both native kidneys and renal allografts. The kidney exhibits a remarkable capability to recover from acute renal failure after IRI⁽²⁰⁾. We found that at 48 hours post IRI, Pacsin 2 is strikingly upregulated in proximal tubules of ischemic kidneys, highlighting the brush-border that is made up of microvilli. Microvillus is composed of a dense bundle of cross-linked actin filaments structure. It increases a cell's surface area, which is especially useful for absorption, secretion and mechanosensation⁽²⁹⁾. Pacsin 2 is known to be enriched at sites with high actin turnover, and to function in the endocytic pathway. Given the weak cytoplasmic expression in proximal tubules in normal adult kidneys, and while not wishing to be bound by any particular theory, the concentrated Pacsin 2 expression at the brush border in ischemic kidneys indicates that Pacsin 2 contribute to both actin nucleation and/or endocytic processes in proximal tubules during kidney repair.

Accordingly, various embodiments of the present invention are based, at least in part, on these finding.

### Diagnosis of Kidney Injuries and Kidney Diseases

Various embodiments of the present invention provide for a method of diagnosing kidney injury or a kidney disease, comprising: assaying a biological sample obtained from a subject to determine a Pacsin 2 expression level; and comparing the Pacsin 2 expression level to a reference level; and diagnosing the kidney injury or a kidney disease if

In various embodiments, the Pacsin 2 expression level is Pacsin 2 protein expression level. In other embodiments, the Pacsin 2 expression level is Pacsin 2 nucleic acid expression level. In certain embodiments, the Pacsin 2 expression level is Pacsin 2 mRNA expression level.

In certain embodiments, the biological sample comprises blood or urine. In certain embodiments, the biological sample comprises a kidney cell, kidney tissue, a brush border of a tubule of a kidney, an apical brush border of a proximal tubule of a kidney, or combinations thereof.

In certain embodiments, the kidney injury or kidney disease is post ischemia-reperfusion injury. In other embodiments, the kidney disease is polycystic kidney disease.

Various embodiments of the present invention provide for a system for diagnosing a kidney injury or kidney disease in a subject in need thereof, comprising: a sample analyzer configured to produce a signal for Pacsin 2 in a biological sample of the subject; and a computer sub-system programmed to calculate, based on the Pacsin 2 level whether the signal is higher than a reference level.

Various embodiments of the present invention provide for a computer program product embodied in a non-transitory computer readable medium that, when executing on a computer, performs steps comprising: detecting a Pacsin 2 expression level in a biological sample from a subject in need of a diagnosis regarding a kidney injury or kidney disease; and comparing the Pacsin 2 expression level to a reference level.

Various embodiments of the present invention provide for a kit for diagnosing a kidney injury or kidney disease in a subject in need thereof, comprising: one or more probes comprising a combination of detectably labeled probes for the detection of Pacsin 2.

In various embodiments, the kit further comprises a computer program product embodied in a non-transitory computer readable medium that, when executing on a computer, performs steps comprising: detecting the Pacsin 2 expression level in a biological sample from a subject in need of a diagnosis regarding a kidney injury or kidney disease; and comparing the Pacsin 2 expression level to a reference level.

### Selecting Therapies for Kidney Injuries and Kidney Diseases

Various embodiments provide for a method of selecting a kidney therapy, and optionally administering the kidney therapy, comprising: assaying a biological sample obtained from a subject to determine a Pacsin 2 expression level; comparing the Pacsin 2 jury or kidney disease if the Pacsin 2 expression level is higher than the reference level; and selecting a kidney therapy if the kidney injury or kidney disease is diagnosed.

In various embodiments, the Pacsin 2 expression level is Pacsin 2 protein expression level. In other embodiments, the Pacsin 2 expression level is Pacsin 2 nucleic acid expression level. In certain embodiments, the Pacsin 2 expression level is Pacsin 2 mRNA expression level.

In various embodiments, the method further comprises administering the kidney therapy if the kidney injury or kidney disease is diagnosed.

In certain embodiments, the biological sample comprises blood or urine. In certain embodiments, the biological sample comprises a kidney cell, kidney tissue, a brush border of a tubule of a kidney, an apical brush border of a proximal tubule of a kidney, or combinations thereof.

In certain embodiments, the kidney injury or kidney disease is post ischemia-reperfusion injury. In other embodiments, the kidney disease is polycystic kidney disease.

In various embodiments, selecting the kidney therapy comprises selecting a therapy from the group consisting of a Pacsin 2 agonist, a Pacsin 2 product, a Pacsin 2 product mimetic and combinations thereof.

In other embodiments selecting the kidney therapy comprises selecting a conventional treatment; for example, those discussed below. The conventional treatments can include those that treat the underlying conditions, as discussed below

In various embodiments, administering the kidney therapy comprises administering a therapy from the group consisting of a Pacsin 2 agonist, a Pacsin 2 product, a Pacsin 2 product mimetic and combinations thereof.

In various embodiments, the Pacsin 2 product comprises a Pacsin 2 protein or an active fragment thereof.

In other embodiments administering the kidney therapy comprises administering a conventional treatment; for example, those discussed below. The conventional treatments can include those that treat the underlying conditions, as discussed below.

Selecting a therapy as used herein, includes but is not limited to selecting, directing, choosing, prescribing, advising, recommending, instructing, or counseling the subject with respect to the therapy.

### Treatment of Kidney Injuries and Kidney Diseases

Various embodiments of the present invention provide for a method of treating a kidney injury or kidney disease, comprising: administering a therapy from the group consisting of a Pacsin 2 agonist, a Pacsin 2 product, a Pacsin 2 product mimetic and combinations thereof to a subject in need thereof.

In various embodiments, the Pacsin 2 product comprises a Pacsin 2 protein or an active fragment thereof.

### Subjects

In various embodiments, the subject undergoing diagnosis, selection of therapy or therapy is a subject who is suspected to have a kidney injury, kidney disease, or has a renal allograft. For example, the subject is exhibiting one or more symptoms indicative of kidney injury, or kidney disease as discussed here.

In certain embodiments, the subject is a mammalian subject. In particular embodiments, the subject is a human subject.

### Human Pacsin 2

The amino acid sequence of human Pacsin 2 is provided herein:

(SEQ ID NO: 1)

MSVTYDDSVGVEVSSDSFWEVGNYKRTVKRIDDGHRLCSDLMNCLHERAR
IEKAYAQQLTEWARRWRQLVEKGPQYGTVEKAWMAFMSEAERVSELHLEV
KASLMNDDFEKIKNWQKEAFHKQMMGGFKETKEAEDGFRKAQKPWAKKLK
EVEAAKKAHHAACKEEKLAISREANSKADPSLNPEQLKKLQDKIEKCKQD
VLKTKEKYEKSLKELDQGTPQYMENMEQVFEQCQQFEEKRLRFFREVLLE
VQKHLDLSNVAGYKAIYHDLEQSIRAADAVEDLRWFRANHGPGMAMNWPQ
FEEWSADLNRTLSRREKKKATDGVTLTGINQTGDQSLPSKPSSTLNVPSN
PAQSAQSQSSYNPFEDEDDTGSTVSEKDDTKAKNVSSYEKTQSYPTDWSD
DESNNPFSSTDANGDSNPFDDDATSGTEVRVRALYDYEGQEHDELSFKAG
DELTKMEDEDEQGWCKGRLDNGQVGLYPANYVEAIQ

In embodiments, where Pacsin 2 protein is selected for treatment or administered for treatment, the Pacsin 2 protein can comprise SEQ ID NO:1. In embodiments, where an active fragment of Pacsin 2 protein is selected for treatment or administered for treatment, the active fragment of Pacsin 2 protein can comprise a fragment of SEQ ID NO:1, wherein the fragment has at least 10, 15, 20, 25, 30, 35, 40, 45, 50, 55, 60, 65, 70, 75, 80, 85, 90, 95, 96, 97, 98, or 99% of the activity of Pacsin 2. In various least or about 25, 50, 75, 100, 150, 200, 250, 300, 350, 400, 410, 420, 430, 440, 450, 460, 470, 475, 476, 477, 478, 479, 480, 481, 482, 483, 484, or 485 contiguous amino acids of SEQ ID NO: 1.

### Biological Samples

Examples of biological samples include but are not limited to cells, tissues, body fluids, whole blood, plasma, stool, intestinal fluids or aspirate, and stomach fluids or aspirate, serum, cerebral spinal fluid (CSF), urine, sweat, saliva, tears, pulmonary secretions, breast aspirate, prostate fluid, seminal fluid, cervical scraping, amniotic fluid, intraocular fluid, mucous, and moisture in breath. In some embodiments, the biological sample is whole blood, blood plasma, blood serum, or urine. In certain embodiments, the biological sample is blood. In certain embodiments, the biological sample is urine. When urine is used as the biological sample, the level of the Pacsin 2 can be normalized to urine creatinine levels or total protein levels. In other embodiments, the level of Pacsin 2 can be normalized to ERK1/2 or β-actin.

In certain embodiments, the biological sample comprises a kidney cell, kidney tissue, an apical brush border of a proximal tubule of a kidney, or combinations thereof. In these embodiments Pacsin 2 expression is detected in the kidney cell, kidney tissue, an apical brush border of a proximal tubule of a kidney, or combinations thereof.

### Kidney Injuries and Kidney Diseases

Kidney injuries and diseases diagnosed and treated by embodiments of the present invention include, but are not limited to, post ischemia-reperfusion injury, polycystic kidney disease, early kidney injury, chronic kidney disease, acute kidney injury, renal ischemia.

Pacsin 2 interacts with polycystin-1, the major polycystic kidney disease (PKD) protein; accordingly, Pacsin 2 can be used as biomarker for kidney diseases, including polycystic kidney disease. Polycystic kidney disease (PKD) is a disorder in which clusters of cysts develop primarily within the kidneys.

Symptoms of PKD include, but are not limited to high blood pressure, back or side pain, headache, increase in the size of the abdomen, blood in the urine, frequent urination, kidney stones, kidney failure, and urinary tract or kidney infections. Accordingly, in various embodiments, one of ordinary skill in the art can further confirm the diagnosis of PKD by the detection of Pacsin 2 with one or more symptoms PKD.

There can be a variety of causes to some of these kidney injuries and diseases (e.g., post ischemia-reperfusion injury, early kidney injury, chronic kidney disease, acute kidney injury, renal ischemia), some of which are discussed below.

Chronic kidney disease includes conditions that damage kidneys and decrease their ability to keep a person healthy. Wastes can build to high levels in the blood and sicken the individual. Complications such as high blood pressure, anemia, weak bones, poor nutritional health and nerve damage can develop. Kidney disease can also increases the risk of having heart and blood vessel disease. These problems can progress slowly over a long period of time. Chronic kidney disease may be caused by diabetes, high blood pressure and other disorders.

Causes of acute kidney injury are numerous and include low blood volume from any cause, exposure to substances harmful to the kidney, and obstruction of the urinary tract. Acute kidney injury can also be caused by disease, crush injury, contrast agents, various antibiotics.

The causes of acute kidney injury can be categorized into prerenal, intrinsic, and postrenal.

Pre-renal causes of acute kidney injury are those that decrease effective blood flow to the kidney. These include systemic causes, such as low blood volume, low blood pressure, heart failure, and local changes to the blood vessels supplying the kidney. The latter include renal artery stenosis, or the narrowing of the renal artery which supplies the kidney with blood, and renal vein thrombosis, which is the formation of a blood clot in the renal vein that drains blood from the kidney.

Renal ischemia ultimately results in functional disorder, depression of GFR, or both. These causes stem from the inadequate cardiac output and hypovolemia or vascular diseases causing reduced perfusion of both kidneys.

Sources of damage to the kidney itself are referred to as intrinsic. Intrinsic acute kidney disease can be due to damage to the glomeruli, renal tubules, or interstitium. Common causes of each include but are not limited to glomerulonephritis, acute tubular necrosis (ATN), and acute interstitial nephritis (AIN). A cause of intrinsic acute renal failure is tumor lysis syndrome.

Post-renal acute kidney injury can be a consequence of urinary tract obstruction. This may be related to benign prostatic hyperplasia, kidney stones, obstructed urinary catheter, bladder stone, bladder, ureteral or renal malignancy.

Acute kidney failure can occur when a person has a condition that slows blood flow to the kidneys, a person experiences direct damage to the kidneys, the kidneys' ureters become blocked and wastes cannot be removed, or there is impaired blood flow to the kidneys.

Diseases and conditions that may slow blood flow to the kidneys and lead to kidney failure include blood or fluid loss, blood pressure medications, heart attack, heart disease, infection, liver failure, use of aspirin, ibuprofen, naproxen, or related drugs, severe allergic reaction (anaphylaxis), severe burns, severe dehydration and damage to the kidneys.

These diseases, conditions and agents may damage the kidneys and lead to acute kidney failure include blood clots in the veins and arteries in and around the kidneys, cholesterol deposits that block blood flow in the kidneys, glomerulonephritis, hemolytic uremic syndrome, a condition that results from premature destruction of red blood cells, infection, lupus (an immune system disorder causing glomerulonephritis), medications, such as certain chemotherapy drugs, antibiotics, dyes used during imaging tests and zoledronic acid, used to treat osteoporosis and hypercalcemia, multiple myeloma, scleroderma, thrombotic thrombocytopenic purpura (TTP), a rare blood disorder, toxins, such as alcohol, heavy metals and cocaine, vasculitis, and urine blockage in the kidneys.

Diseases and conditions that block the passage of urine out of the body (urinary obstructions) and can lead to acute kidney failure include bladder cancer, blood clots in the urinary tract, cervical cancer, colon cancer, enlarged prostate, kidney stones, nerve damage involving the nerves that control the bladder and prostate cancer.

### Assays

The assays used in the methods, systems and kits described herein can be assays known in the art. In various embodiments, the assays are assays as described herein.

### Protein and Peptide Detection

One of ordinary skill in the art will readily appreciate methods and systems that can be used to detect the Pacsin expression level. These methods and systems include but are not limited to enzyme-linked immunosorbent assay (ELISA), immunohistochemistry, flow cytometry, fluorescence in situ hybridization (FISH), radioimmuno assays, and affinity purification. Examples of ELISAs include but are not limited to indirect ELISA, sandwich ELISA, competitive ELISA, multiple and portable ELISA. In various embodiments, ISA.

In various embodiments of the methods, systems and kits described herein, the assay is an assay to detect the Pacsin 2 expression level. The assay can comprise: a first reagent (e.g., a capture antibody) to react with the Pacsin 2 in the biological sample if the biological sample comprises the Pacsin 2 (if Pacsin 2 is not present, then the first reagent will not react with the Pacsin 2 in the biological sample, but the first reagent is still present in the assay), a second reagent (e.g., a detecting antibody) to react with the Pacsin 2, a third reagent (e.g., a secondary antibody) to react with the second reagent and a substrate (e.g., to react with a label on the third reagent and produce a signal). In various embodiments, the third reagent comprises a label to produce a signal to indicate the presence and/or level of the Pacsin 2. In various embodiments, the label is a radiolabel, a chromophore, a fluorophore, a quantum dot, an enzyme, horseradish peroxidase (HRP), an alkaline phosphatase (AP), biotin, or a combination thereof. In various embodiments, the label is an enzyme that will react with the substrate. In various embodiments, the first reagent is on a solid phase (e.g., plate, multi-well plate).

In various embodiments, the first reagent, second reagent comprising a label, and substrate are all on one solid phase (e.g., dipstick). In a further embodiment, the first reagent, second reagent comprising a label, substrate, as well as control reagents, are all on one solid phase (e.g., dipstick).

In various embodiments, the assay comprises a solid phase; a first reagent to react with Pacsin 2, wherein the first reagent is immobilized on the solid phase; a second reagent to specifically react with Pacsin 2, wherein the second reagent comprise a label; a substrate to react with the label; a third reagent to react with the second reagent to serve as a control. In various embodiments, the solid phase is a capillary membrane. When used, two bands (circles or the like) can indicate a positive result (e.g., the patient kidney injury or kidney disease; and one band (circles or the like) can indicate a negative result. In various embodiments, the first reagent can be an antibody that specifically binds to the Pacsin 2. In various embodiments, the control region or a further control region of the assay can be configured to produce a signal for comparing the test region to the control region such that one can determine if the test sample has a higher or lower level of Pacsin 2.

In various embodiments the substrate is a chromogenic substrate (e.g., 3,3′,5,5′-Tetramethylbenzidine (TMB), 3,3′-Diaminobenzidine (DAB), 2,2′-azino-bis(3-ethylbenzothiazoline-6-sulphonic acid) (ABTS). In various embodiments, the substrate is a chemiluminescence substrate (e.g., ECL).

In various embodiments, the assay to detect the Pacsin 2 expression level also comprises a control.

### Nucleic Acid Detection

### A. Nucleic Acid Isolation

Nucleic acid samples derived from biological samples of a subject that can be used in the methods of the invention to determine the Pacsin 2 expression level in the bioligcal sample can be prepared by means well known in the art. For example, surgical procedures or needle biopsy aspiration can be used to collect cell or tissue samples from a subject. In some embodiments, it is important to enrich and/or purify the biological sample that is tested from the original sample obtained. In other embodiments, the biological sample can then be microdissected to reduce the amount of normal tissue contamination prior to extraction of genomic nucleic acid or pre-RNA for use in the methods of the invention. In still another embodiment, the biological sample are enriched for cells of interest by at least 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range in between, in target cell content. Such enrichment can be accomplished according to methods well-known in the art, such as needle microdissection, laser microdissection, fluorescence activated cell sorting, and immunological cell sorting. In one embodiment, an automated machine performs the cell enrichment to thereby transform the biological sample into a purified form enriched for the presence of cells of interest (e.g., kidney cells).

Collecting nucleic acid samples from control cells of a subject can also be accomplished with surgery or aspiration. In certain embodiments of the methods of the invention, nucleic acid samples from control tissues are not derived from the same tissue type as the tissue and/or cells of interest sampled, and/or are not derived from the patient. The nucleic acid samples from control tissues may be derived from any disease-free tissue and/or cells. Such control samples can be collected by surgical or non-surgical procedures. In certain embodiments, control nucleic acid samples are derived from injury free or disease free tissues.

In one embodiment, the nucleic acid samples used to compute a reference value are taken from at least 1, 2, 5, 10, 20, 30, 40, 50, 100, or 200 different organisms of that species. According to certain aspects of the invention, nucleic acid “derived from” genomic DNA, as used in the methods of the invention, e.g., in hybridization experiments to determine acid generated by restriction enzyme digestion and/or ligation to other nucleic acid, and/or amplification products of genomic nucleic acids, or pre-messenger RNA (pre-mRNA), amplification products of pre-mRNA, or genomic DNA fragments grown up in cloning vectors generated, e.g., by “shotgun” cloning methods. In certain embodiments, genomic nucleic acid samples are digested with restriction enzymes.

### B. Amplification of Nucleic Acids

Though the nucleic acid sample need not comprise amplified nucleic acid, in some embodiments, the isolated nucleic acids can be processed in manners requiring and/or taking advantage of amplification. The genomic DNA samples of a subject optionally can be fragmented using restriction endonucleases and/or amplified prior to determining analysis. In one embodiment, the DNA fragments are amplified using polymerase chain reaction (PCR). Methods for practicing PCR are well known to those of skill in the art. One advantage of PCR is that small quantities of DNA can be used. For example, genomic DNA from a subject may be about 150 ng, 175, ng, 200 ng, 225 ng, 250 ng, 275 ng, or 300 ng of DNA.

In certain embodiments of the methods of the invention, the nucleic acid from a subject is amplified using a single primer pair. For example, genomic DNA samples can be digested with restriction endonucleases to generate fragments of genomic DNA that are then ligated to an adaptor DNA sequence which the primer pair recognizes. In other embodiments of the methods of the invention, the nucleic acid of a subject is amplified using sets of primer pairs specific to Pacsin 2. Such sets of primer pairs each recognize genomic DNA sequences flanking Pacsin 2. A DNA sample suitable for hybridization can be obtained, e.g., by polymerase chain reaction (PCR) amplification of genomic DNA, fragments of genomic DNA, fragments of genomic DNA ligated to adaptor sequences or cloned sequences. Computer programs that are well known in the art can be used in the design of primers with the desired specificity and optimal amplification properties, such as Oligo version 5.0 (National Biosciences). PCR methods are well known in the art, and are described, for example, in Innis et al., eds., 1990, PCR Protocols: A Guide to Methods And Applications, Academic Press Inc., San Diego, Calif. It will be apparent to one skilled in the art that controlled robotic systems are useful for isolating and amplifying nucleic acids and can be used.

In other embodiments, where genomic DNA of a subject is fragmented using restriction endonucleases and amplified prior to analysis, the amplification can comprise cloning regions of genomic DNA of the subject. In such methods, amplification of the DNA regions is achieved through the cloning process. For example, expression vectors can be engineered to express large quantities of particular fragments of genomic DNA of the subject.

In yet other embodiments, where the DNA of a subject is fragmented using restriction endonucleases and amplified prior to analysis, the amplification comprises expressing a nucleic acid encoding a gene, or a gene and flanking genomic regions of nucleic acids, from the subject. RNA (pre-messenger RNA) that comprises the entire transcript including introns is then isolated and used in the methods of the invention to analyze and provide a genetic signature of a the biological sample (e.g., injured and/or diseased kidney cells or tissue). In certain embodiments, no amplification is required. In such embodiments, the genomic DNA, or pre-RNA, of a subject may be fragmented using restriction endonucleases or other methods. The resulting fragments may be hybridized to SNP probes. Typically, greater quantities of DNA are needed to be isolated in comparison to the quantity of DNA or pre-mRNA needed where fragments are amplified. For example, where the nucleic acid of a subject is not amplified, a DNA sample of a subject for use in hybridization may be about 400 ng, 500 ng, 600 ng, 700 ng, 800 ng, 900 ng, or 1000 ng of DNA or greater. Alternatively, in other embodiments, methods are used that require very small amounts of nucleic acids for analysis, such as less than 400 ng, 300 ng, 200 ng, 100 ng, 90 ng, 85 ng, 80 ng, 75 ng, 70 ng, 65 ng, 60 ng, 55 ng, 50 ng, or less, such as is used for molecular inversion probe (MIP) assays. These techniques are particularly useful for analyzing clinical samples, such as paraffin embedded formalin-fixed material or small core needle biopsies, characterized as being readily available but generally having reduced DNA quality (e.g., small, fragmented DNA) and/or not providing large amounts of nucleic acids.

### C. Hybridization

The nucleic acid samples derived from a subject used in the methods of the invention can be hybridized to arrays comprising probes (e.g., oligonucleotide probes) in order to identify Pacsin 2. In some embodiments, the probes used in the methods of the invention comprise an array of probes that can be tiled on a DNA chip (e.g., SNP oligonucleotide probes). In some embodiments, Pacsin 2 expression is determined by a method that does not comprise detecting a change in size of restriction enzyme-digested nucleic acid fragments. In other embodiments, SNPs are analyzed to identify Pacsin 2 expression. Hybridization and wash conditions used in the methods of the invention are chosen so that the nucleic acid samples to be analyzed by the invention specifically bind or specifically hybridize to the complementary oligonucleotide sequences of the array, ry DNA is located. In some embodiments, the complementary DNA can be completely matched or mismatched to some degree as used, for example, in Affymetrix oligonucleotide arrays such as those used to analyze SNPs in MIP assays. The single-stranded synthetic oligodeoxyribonucleic acid DNA probes of an array may need to be denatured prior to contact with the nucleic acid samples from a subject, e.g., to remove hairpins or dimers which form due to self-complementary sequences.

Optimal hybridization conditions will depend on the length of the probes and type of nucleic acid samples from a subject. General parameters for specific (i.e., stringent) hybridization conditions for nucleic acids are described and known in the art.

### D. Oligonucleotide Nucleic Acid Arrays

Various formats of DNA arrays that employ oligonucleotide “probes,” (i.e., nucleic acid molecules having defined sequences) are well known to those of skill in the art. Typically, a set of nucleic acid probes, each of which has a defined sequence, is immobilized on a solid support in such a manner that each different probe is immobilized to a predetermined region. In certain embodiments, the set of probes forms an array of positionally-addressable binding (e.g., hybridization) sites on a support. Each of such binding sites comprises a plurality of oligonucleotide molecules of a probe bound to the predetermined region on the support. More specifically, each probe of the array is preferably located at a known, predetermined position on the solid support such that the identity (i.e., the sequence) of each probe can be determined from its position on the array (i.e., on the support or surface). Microarrays can be made in a number of ways, of which several are described herein. However produced, microarrays share certain characteristics, they are reproducible, allowing multiple copies of a given array to be produced and easily compared with each other.

Preferably, the microarrays are made from materials that are stable under binding (e.g., nucleic acid hybridization) conditions. The microarrays are preferably small, e.g., between about 1 cm² and 25 cm², preferably about 1 to 3 cm². However, both larger and smaller arrays are also contemplated and may be preferable, e.g., for simultaneously evaluating a very large number of different probes. Oligonucleotide probes can be synthesized directly on a support to form the array. The probes can be attached to a solid support or surface, which may be made, e.g., from glass, plastic (e.g., polypropylene, nylon), polyacrylamide, nitrocellulose, gel, or other porous or nonporous material. The set of immobilized probes or the array of immobilized probes is contacted with a sample containing quences complementary to an immobilized probe hybridize or bind to the probe. After separation of, e.g., by washing off, any unbound material, the bound, labeled sequences are detected and measured. The measurement is typically conducted with computer assistance. Using DNA array assays, complex mixtures of labeled nucleic acids, e.g., nucleic acid fragments derived a restriction digestion of genomic DNA from the biological sample, can be analyzed. DNA array technologies have made it possible to determine the expression level of Pacsin 2.

In certain embodiments, high-density oligonucleotide arrays are used in the methods of the invention. These arrays containing thousands of oligonucleotides complementary to defined sequences, at defined locations on a surface can be synthesized in situ on the surface by, for example, photolithographic techniques (see, e.g., Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; 5,510,270; 5,445,934; 5,744,305; and 6,040,138). Methods for generating arrays using inkjet technology for in situ oligonucleotide synthesis are also known in the art (see, e.g., Blanchard, International Patent Publication WO 98/41531, published Sep. 24, 1998; Blanchard et al., 1996, Biosensors And Bioelectronics 11:687-690; Blanchard, 1998, in Synthetic DNA Arrays in Genetic Engineering, Vol. 20, J. K. Setlow, Ed., Plenum Press, New York at pages 111-123). Another method for attaching the nucleic acids to a surface is by printing on glass plates, as is described generally by Schena et al. (1995, Science 270:467-470). Other methods for making microarrays, e.g., by masking (Maskos and Southern, 1992, Nucl. Acids. Res. 20:1679-1684), may also be used. When these methods are used, oligonucleotides (e.g., 15 to 60-mers) of known sequence are synthesized directly on a surface such as a derivatized glass slide. The array produced can be redundant, with several oligonucleotide molecules corresponding to each informative locus of interest (e.g., SNPs, RFLPs, STRs, etc.).

One exemplary means for generating the oligonucleotide probes of the DNA array is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 15 and about 600 bases in length, more typically between about 20 and about 100 bases, most preferably between about 40 and about 70 bases in length. In some embodiments, synthetic nucleic acids include non-natural bases, such as, but by no means limited to, inosine. As noted above, nucleic acid analogues may be used as binding nalogue is peptide nucleic acid (see, e.g., Egholm et al., 1993, Nature 363:566-568; U.S. Pat. No. 5,539,083). In alternative embodiments, the hybridization sites (i.e., the probes) are made from plasmid or phage clones of regions of genomic DNA corresponding to SNPs or the complement thereof. The size of the oligonucleotide probes used in the methods of the invention can be at least 10, 20, 25, 30, 35, 40, 45, or 50 nucleotides in length. It is well known in the art that although hybridization is selective for complementary sequences, other sequences which are not perfectly complementary may also hybridize to a given probe at some level. Thus, multiple oligonucleotide probes with slight variations can be used, to optimize hybridization of samples. To further optimize hybridization, hybridization stringency condition, e.g., the hybridization temperature and the salt concentrations, may be altered by methods that are well known in the art.

In some embodiments, the high-density oligonucleotide arrays used in the methods of the invention comprise oligonucleotides corresponding to Pacsin 2. The oligonucleotide probes may comprise DNA or DNA “mimics” (e.g., derivatives and analogues) corresponding to a portion of each informative locus of interest (e.g., SNPs, RFLPs, STRs, etc.) in a subject's genome. The oligonucleotide probes can be modified at the base moiety, at the sugar moiety, or at the phosphate backbone. Exemplary DNA mimics include, e.g., phosphorothioates. For each SNP locus, a plurality of different oligonucleotides may be used that are complementary to the sequences of sample nucleic acids. For example, for a single informative locus of interest (e.g., SNPs, RFLPs, STRs, etc.) about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more different oligonucleotides can be used. Each of the oligonucleotides for a particular informative locus of interest may have a slight variation in perfect matches, mismatches, and flanking sequence around the SNP. In certain embodiments, the probes are generated such that the probes for a particular informative locus of interest comprise overlapping and/or successive overlapping sequences which span or are tiled across a genomic region containing the target site, where all the probes contain the target site. By way of example, overlapping probe sequences can be tiled at steps of a predetermined base interval, e. g. at steps of 1, 2, 3, 4, 5, 6, 7, 8, 9, or 10 bases intervals. In certain embodiments, the assays can be performed using arrays suitable for use with molecular inversion probe protocols such as described by Wang et al. (2007) Genome Biol. 8, R246. For oligonucleotide probes targeted at nucleic acid species of closely resembled (i.e., homologous) sequences, “cross-hybridization” among similar probes can zation measurements. Cross-hybridization is a particularly significant concern in the detection of SNPs since the sequence to be detected (i.e., the particular SNP) must be distinguished from other sequences that differ by only a single nucleotide. Cross-hybridization can be minimized by regulating either the hybridization stringency condition and/or during post-hybridization washings. Highly stringent conditions allow detection of allelic variants of a nucleotide sequence, e.g., about 1 mismatch per 10-30 nucleotides. There is no single hybridization or washing condition which is optimal for all different nucleic acid sequences. For particular arrays of Pacsin 2 these conditions can be identical to those suggested by the manufacturer or can be adjusted by one of skill in the art. In some embodiments, the probes used in the methods of the invention are immobilized (i.e., tiled) on a glass slide called a chip. For example, a DNA microarray can comprises a chip on which oligonucleotides (purified single-stranded DNA sequences in solution) have been robotically printed in an (approximately) rectangular array with each spot on the array corresponds to a single DNA sample which encodes an oligonucleotide. In summary the process comprises, flooding the DNA microarray chip with a labeled sample under conditions suitable for hybridization to occur between the slide sequences and the labeled sample, then the array is washed and dried, and the array is scanned with a laser microscope to detect hybridization. In certain embodiments there are at least 250, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 50,000, 60,000, 70,000, 80,000, 90,000, 100,000 or more or any range in between, of Pacsin 2 for which probes appear on the array (with match/mismatch probes for a single locus of interest or probes tiled across a single locus of interest counting as one locus of interest). The maximum number of Pacsin 2 being probed per array is determined by the size of the genome and genetic diversity of the subjects species. DNA chips are well known in the art and can be purchased in pre-5 fabricated form with sequences specific to particular species. In some embodiments, the Genome-Wide Human SNP Array 6.0™ and/or the 50K XbaI arrays (Affymetrix, Santa Clara, Calif.) are used in accordance with various methods of the invention. In other embodiments, SNPs can be detected and quantitated using sequencing methods, such as “next-generation sequencing methods”.

### E. Signal Detection

In some embodiments, nucleic acid samples derived from a subject are certain embodiments, nucleic acid samples derived from each of the two sample types of a subject (i.e., suspected injury/disease cells and tissues and disease and injury free cells and tissue) are hybridized to separate, though identical, arrays. In certain embodiments, nucleic acid samples derived from one of the two sample types of a subject (i.e., suspected injury/disease cells and tissues and disease and injury free cells and tissue) is hybridized to such an array, then following signal detection the chip is washed to remove the first labeled sample and reused to hybridize the remaining sample. In other embodiments, the array is not reused more than once. In certain embodiments, the nucleic acid samples derived from each of the two sample types of a (i.e., suspected injury/disease cells and tissues and disease and injury free cells and tissue) are differently labeled so that they can be distinguished. When the two samples are mixed and hybridized to the same array, the relative intensity of signal from each sample is determined for each site on the array, and any relative difference in abundance of Pacsin 2. Signals can be recorded and, in some embodiments, analyzed by computer. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the array, a ratio of the emission of the two fluorophores can be calculated, which may help in eliminating cross hybridization signals to more accurately determining whether a particular SNP locus is heterozygous or homozygous.

### F. Labeling

In some embodiments, the nucleic acids samples, fragments thereof, or fragments thereof ligated to adaptor regions used in the methods of the invention are detectably labeled. For example, the detectable label can be a fluorescent label, e.g., by incorporation of nucleotide analogues. Other labels suitable for use in the present invention include, but are not limited to, biotin, iminobiotin, antigens, cofactors, dinitrophenol, lipoic acid, olefinic compounds, detectable polypeptides, electron rich molecules, enzymes capable of generating a detectable signal by action upon a substrate, and radioactive isotopes.

Radioactive isotopes include that can be used in conjunction with the methods of the invention, but are not limited to, ³²P and ¹⁴C. Fluorescent molecules suitable for the present invention include, but are not limited to, fluorescein and its derivatives, rhodamine and its derivatives, texas red, 5′carboxy-fluorescein (“FAM”), 2′,7′-dimethoxy-4′,5′-dichloro-6-carboxy-fluorescein (“JOE”), N,N,N′,N′-tetramethyl-6-carboxy-rhodamine RD40, and IRD41.

Fluorescent molecules which are suitable for use according to the invention further include: cyamine dyes, including but not limited to Cy2, Cy3, Cy3.5, CY5, Cy5.5, Cy7 and FLUORX; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold.

Two-color fluorescence labeling and detection schemes may also be used (Shena et al., 1995, Science 270:467-470). Use of two or more labels can be useful in detecting variations due to minor differences in experimental conditions (e.g., hybridization conditions). In some embodiments of the invention, at least 5, 10, 20, or 100 dyes of different colors can be used for labeling. Such labeling would also permit analysis of multiple samples simultaneously which is encompassed by the invention.

The labeled nucleic acid samples, fragments thereof, or fragments thereof ligated to adaptor regions that can be used in the methods of the invention are contacted to a plurality of oligonucleotide probes under conditions that allow sample nucleic acids having sequences complementary to the probes to hybridize thereto. Depending on the type of label used, the hybridization signals can be detected using methods well known to those of skill in the art including, but not limited to, X-Ray film, phosphor imager, or CCD camera. When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al. (1996) Genome Res. 6, 639-645). In a preferred embodiment, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al. (1996) Genome Res. 6, 639-645. Alternatively, a fiber-optic bundle can be used such as that described by Ferguson et al. (1996) Nat. Biotech. 14, 1681-1684. The resulting signals can then be) outer software.

### Reference Levels

In various embodiments of the present invention, the reference level is based on the normal level or normal range of subject who does not have kidney injury or kidney disease. In certain embodiments, a Pacsin 2 expression level higher than the reference level is indicative of kidney injury or kidney disease. In these embodiments, the Pacsin 2 expression level can be increased by at least or about 5, 10, 20, 30, 40, 50, 60, 70, 80, or 90% compared to reference level to result in a diagnosis of kidney injury or kidney disease. In various embodiments, the Pacsin 2 expression level can be increased by at least or about 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold 2.2-fold 2.3-fold 2.4-fold 2.5-fold, 2.6-fold, 2.7-fold, 2.8-fold, 2.9-fold, or 3-fold, 4-fold, 5-fold, 6-fold, 7-fold, 8-fold, 9-fold or 10-fold compared to reference level.

In other embodiments, the reference level is the average reference level for the Pacsin 2 from a population of healthy subjects. In other embodiments, the reference level is the average plus one or two standard deviations of the Pacsin 2 expression level from a population of healthy subjects (e.g., reference range). In some embodiments, the population of healthy subjects can range from at least three healthy individuals to 25 healthy individuals, and even more than 50 healthy individuals.

### Non-Human Machines/Computer Implementation Systems and Methods

Various embodiments of the present invention provides for a non-transitory computer readable medium comprising instructions to execute the methods of the present invention, as described herein.

In certain embodiments, the methods of the invention implement a computer program for example, to compare the Pacsin 2 expression level. For example, a non-transitory computer program can be used.

Numerous types of computer systems can be used to implement the analytic methods of this invention according to knowledge possessed by a skilled artisan in the bioinformatics and/or computer arts.

Several software components can be loaded into memory during operation of such a computer system. The software components can comprise both software components that are standard in the art and components that are special to the present invention. The methods of the invention can also be programmed or modeled in mathematical software el specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.). In certain embodiments, the computer comprises a database for storage of Pacsin 2 expression levels. Such stored profiles can be accessed and used to compare Pacsin 2 expression levels in the sample to known control/reference levels.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

Once a laboratory technician or laboratory professional or group of laboratory technicians or laboratory professionals determines the Pacsin 2 expression level, the same or a different laboratory technician or laboratory professional (or group) can analyze one or more assays to determine whether the Pacsin 2 expression level differs from the reference level or reference range, and then determine that the subject has kidney injury or kidney disease if the Pacsin 2 expression levels do differ.

In various embodiments, provided herein is a non-transitory computer readable storage medium comprising: a storing data module containing data from a sample comprising a Pacsin 2 expression level; a detection module to detect the Pacsin 2 expression level; a comparison module that compares the data stored on the storing data module with a reference data and/or control data, and to provide a comparison content, and an output module displaying the comparison content for the user, wherein an indication that the subject has kidney injury or kidney diseaes is displayed when the Pacsin 2 expression level differs from the reference level. In various embodiments, the reference level is a reference range.

In various embodiments, the control data comprises data from patients who do not have kidney injury or kidney disease.

Embodiments of the invention can be described through functional modules, which are defined by computer executable instructions recorded on a non-transitory computer readable media and which cause a computer to perform method steps when executed. The modules are segregated by function, for the sake of clarity. However, it should be understood that the modules/systems need not correspond to discreet blocks of code and the described functions can be carried out by the execution of various code portions stored on various e appreciated that the modules may perform other functions, thus the modules are not limited to having any particular functions or set of functions.

The non-transitory computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can be accessed by a computer including and any suitable combination of the foregoing.

Computer-readable data embodied on one or more non-transitory computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or y methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine 2^{nd} ed. (CRC Press, London, 2005) and Ouelette and Bzevanis Bioinformatics: A Practical Guide for Analysis of Gene and Proteins (Wiley & Sons, Inc., 3^{rd} ed., 2004).

The functional modules of certain embodiments of the invention, include for example, a measuring module, a storage module, a comparison module, and an output module. The functional modules can be executed on one, or multiple, computers, or by using one, or multiple, computer networks. The measuring module has computer executable instructions to provide, e.g., expression information in computer readable form.

The measuring module, can comprise any system for detecting the Pacsin 2 expression level.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon the Pacsin 2 expression level information. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising Pacsin 2 expression level information.

In one embodiment the reference data stored in the storage module to be read by the comparison module is, e.g., data from patients who do not have kidney injury or kidney disease.

The “comparison module” can use a variety of available software programs and formats for the comparison operative to compare binding data determined in the measuring module to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related, for example, Pacsin 2 expression levels.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content-based in part on the comparison result that may be stored and output as requested by a user using an output module.

The content based on the comparison result, may be Pacsin 2 expression levels compared to reference levels.

In various embodiments of the invention, the content based on the comparison result is displayed on a computer monitor. In various embodiments of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

### Treatments

### Pacsin 2 Treatments

Treatment of kidney injury, including early kidney injury, post ischemia reperfusion injury or kidney diseases can include using a Pacsin 2 agonist, a Pacsin 2 product, a Pacsin 2 product mimetic or combinations thereof.

As used herein, the term “Pacsin 2 agonist” refers to a compound/composition which achieves at least 10% (e.g., 10%, 15%, 20%, 25%, 30%, 35%, 40%, 45%, 50%, 55%, 60%, 65%, 70%, 75%, 80%, 85%, 90%, 95% or more) activation of Pacsin 2. Without limitations, a Pacsin 2 agonist can be selected from the group consisting of small organic or inorganic molecules; saccharines; oligosaccharides; polysaccharides; biological macromolecules; peptides; proteins; peptide analogs and derivatives; peptidomimetics; antibodies; antigen binding fragments of antibodies; nucleic acids, e.g., oligonucleotides, antisense oligonucleotides, ribozymes, aptamers, microRNAs, pre-microRNAs, plasmid atives; an extract made from biological materials such as bacteria, plants, fungi, or animal cells; animal tissues; naturally occurring or synthetic compositions; and any combinations thereof. It is noted that the term “Pacsin 2 agonist” as used herein comprises naturally occurring botanical sources that include Pacsin 2 activator compounds or components.

Pacsin 2 product includes the Pacsin 2 protein, active fragments thereof and functional fragments thereof.

Methods of designing peptide mimetics and screening of functional peptide mimetics are well known in the art. One basic method of designing a molecule which mimics a known protein or peptide, is first to identifies the active region(s) of the known protein (for example in the case of an antibody-antigen interaction one identifies which region(s) of the antibody enable binding to the antigen), and then searches for a mimetic which emulates the active region. Since the active region of the known protein is relatively small, it is hoped that a mimetic will be found which is much smaller (e.g. in molecular weight) than the protein, and correspondingly easier and cheaper to synthesize. Such a mimetic could be used as a convenient substitute for the protein, as an agent for interacting with the target molecule.

For example, Reineke et al. (1999, Nature Biotechnology, 17; 271-275) designed a mimic molecule which mimics a binding site of the interleukin-10 protein using a large library of short peptides were synthesized, each of which corresponded to a short section of interleukin 10. The binding of each of these peptides to the target (in this case an antibody against interleukin-10) was then tested individually by an assay technique, to identify potentially relevant peptides. Phage display libraries of peptides and alanine scanning method can be used.

Other methods for designing peptide mimetic to a particular peptide or protein include European Patent EP1206494, the SuperMimic program by Andrean Goede et. al. 2006 BMC Bioinformatics, 7:11; and MIMETIC program by W. Campbell et. al., 2002, Microbiology and Immunology 46:211-215. The SuperMimic program is designed to identify compounds that mimic parts of a protein, or positions in proteins that are suitable for inserting mimetics. The application provides libraries that contain peptidomimetic building blocks on the one hand and protein structures on the other. The search for promising peptidomimetic linkers for a given peptide is based on the superposition of the peptide with several conformers of the mimetic. New synthetic elements or proteins can be imported and used for searching. The MIMETIC computer program, which generates a series of peptides for interaction with a target peptide sequence is taught by W. Campbell et. al., 2002. In depth s Design with the Aid of Computational Chemistry” by James R. Damewood Jr. in Reviews in Computational Chemistry Reviews in Computational Chemistry, January 2007, Volume 9 Book Series: Reviews in Computational Chemistry, Editor(s): Kenny B. Lipkowitz, Donald B. BoydPrint ISBN: 9780471186397 ISBN: 9780470125861 Published by John Wiley &Sons, Inc.; and in T. Tselios, et. al., Amino Acids, 14: 333-341, 1998.

Methods for preparing libraries containing diverse populations of peptides, peptoids and peptidomimetics are well known in the art and various libraries are commercially available (see, for example, Ecker and Crooke, Biotechnology 13:351-360 (1995), and Blondelle et al., Trends Anal. Chem. 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37:1385-1401 (1994), each of which is incorporated herein by reference). One skilled in the art understands that a peptide can be produced in vitro directly or can be expressed from a nucleic acid, which can be produced in vitro. Methods of synthetic peptide and nucleic acid chemistry are well known in the art.

A library of peptide molecules also can be produced, for example, by constructing a cDNA expression library from mRNA collected from a tissue of interest. Methods for producing such libraries are well known in the art (see, for example, Sambrook et al., Molecular Cloning: A laboratory manual (Cold Spring Harbor Laboratory Press), which is incorporated herein by reference). Preferably, a peptide encoded by the cDNA is expressed on the surface of a cell or a virus containing the cDNA.

### Conventional Treatments

In cases of polycystic kidney disease, a goal of treatment is to control symptoms and prevent complications. Treatments for PKD can include, but are not limited to blood pressure medicines, diuretics, low-salt diet, and antibiotics for any urinary tract infections. Cysts that are painful, infected, bleeding, or causing a blockage can be drained. In some cases, surgery to remove one or both kidneys may be needed. Treatments for end-stage PKD disease may include dialysis or a kidney transplant.

The management of acute kidney injury can include the treatment of the underlying cause. In addition to treatment of the underlying disorder, management of acute kidney injury routinely includes the avoidance of substances that are toxic to the kidneys, n, iodinated contrasts such as those used for CT scans, many antibiotics such as gentamicin, and a range of other substances.

Monitoring of renal function, by serial serum creatinine measurements and monitoring of urine output, is routinely performed. In the hospital, insertion of a urinary catheter helps monitor urine output and relieves possible bladder outlet obstruction, such as with an enlarged prostate.

Specific therapies for pre renal acute kidney injury include administration of intravenous fluids to improve renal function. Volume status may be monitored with the use of a central venous catheter to avoid over- or under-replacement of fluid.

If low blood pressure is a persistent problem in a fluid-replete patient, inotropes such as norepinephrine and dobutamine may be given to improve cardiac output and hence renal perfusion.

The causes of intrinsic acute kidney injury require specific therapies. For example, intrinsic acute kidney injury due to Wegener's granulomatosis may respond to steroid medication. Toxin-induced prerenal AKI often responds to discontinuation of the offending agent, such as aminoglycoside, penicillin, NSAIDs, or paracetamol.

If the cause is obstruction of the urinary tract, relief of the obstruction; for example, with a nephrostomy or urinary catheter, may be necessary.

The use of diuretics such as furosemide, is widespread and sometimes convenient in ameliorating fluid overload, and is not associated with higher mortality.

Renal replacement therapy, such as with hemodialysis, may be instituted in some cases of acute kidney injury.

Lack of improvement with fluid resuscitation, therapy-resistant hyperkalemia, metabolic acidosis, or fluid overload may necessitate artificial support in the form of dialysis or hemofiltration.

Treatment for acute kidney failure involves identifying the illness or injury that originally damaged kidneys.

If acute kidney failure is caused by a lack of fluids in the blood, intravenous (IV) fluids can be used. In other cases, wherein acute kidney failure may causes excess fluid, leading to swelling in arms and legs, diuretics can be used.

If the kidneys are not properly filtering potassium from blood calcium, glucose or sodium polystyrene sulfonate (Kayexalate, Kionex) can be used to prevent the accumulation of high levels of potassium the blood. Too much potassium in the blood can weakness.

If the levels of calcium in the blood drop too low, an infusion of calcium can be performed.

If toxins build up in the blood, hemodialysis (also referred to as dialysis) may be to help remove toxins and excess fluids from the body while the kidneys heal. Dialysis may also help remove excess potassium from the body.

### Formulation and Administration

In various embodiments, the kidney therapy is delivered in a pharmaceutically acceptable carrier in a therapeutically effective amount.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990) or Remington: The Science and Practice of Pharmacy, 22^{nd} Ed., Loyd et al., ed. (Pharmaceutical Press 2012). The formulation should suit the mode of administration. Additional carrier agents, such as liposomes, can be added to the pharmaceutically acceptable carrier.

As used herein, the term “a therapeutically effective amount” refers an amount sufficient to achieve the intended purpose. For example, an effective amount of a kidney therapy will cause a reduction or even completely halt one or more symptoms of ing or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

As used herein, the terms “administering,” refers to the placement of a kidney therapy into a subject by a method or route which results in at least partial localization of the kidney therapy at one or more desired site(s). The kidney therapy can be administered by any appropriate route which results in an effective treatment in the subject. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

Therapeutic compositions contain a physiologically tolerable carrier together with an active agent as described herein, dissolved or dispersed therein as an active ingredient. In one embodiment, the therapeutic composition is not immunogenic when administered to a mammal or human patient for therapeutic purposes. As used herein, the e” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a the disorder or condition, and can be determined by standard clinical techniques. In one embodiment, the term “pharmaceutically acceptable” means approved by a regulatory agency of the Federal or a state government or listed in the U.S. Pharmacopeia or other generally recognized pharmacopeia for use in animals, and more particularly in humans.

The precise dose and formulation to be employed depends upon the potency of the agent, and include amounts large enough to produce the desired effect, e.g., a reduction in one or more symptoms of kidney injury or kidney disease. The dosage should not be so large as to cause unacceptable adverse side effects. Generally, the dosage will vary with the type of kidney therapy, and with the age, condition, and sex of the patient are also considered. Dosage and formulation of the kidney therapy will also depend on the route of administration, and the seriousness and/or extent of the disease or disorder, and should be decided according to the judgment of the practitioner and each patient's circumstances. Effective doses can be extrapolated from dose-response curves derived from in vitro or animal model test systems.

The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In various embodiments, the doses recited above are administered daily for several weeks or months. The duration of treatment depends upon the subject's clinical progress and responsiveness to therapy. Continuous, relatively low maintenance doses are contemplated after an initial higher therapeutic dose.

Efficacy testing can be performed during the course of treatment using the methods described herein. Measurements of the degree of severity of a number of symptoms associated with a particular ailment are noted prior to the start of a treatment and then at later specific time period after the start of the treatment.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Materials and Methods

### Cell Culture

Mouse inner medullary collecting duct cells (mIMCD3) were purchased from ATCC (CRL-2123) and cultured in DMEM/F12 50/50 supplemented with 10% fetal bovine serum.

Establishment of Stable Pacsin 2 Knockdown mIMCD3 Cell Lines

For the creation of stable cell lines expressing Pacsin 2 shRNA, the pSilencer 2.1-U6-Neo-Pacsin 2 shRNA plasmid was transfected into mIMCD3 cells with lipofectamine 2000 (Invitrogen) by following the manufacturer's instructions. The mouse Pacsin 2 shRNA target sequence is 5′-ATGTCTGTCACCT ACGATG-3′ (SEQ ID NO:2). As a control, the pSilencer 2.1-U6-Neo plasmid (Ambion) encoding a small hairpin shRNA which shows no homology to any known gene, was also transfected into cells. Cells were selected using 1 mg/ml G418 in growth media for 20 d. Surviving clones were transferred onto a 96-well plate and when confluent, transferred to 24-, 12-, and six-well plates. The knockdown efficiency was confirmed by western blotting and semi-quantitative-PCR analyses.

### Western Analysis

Briefly, the protein samples were separated on SDS-acrylamide resolving gels and transferred to Hybond ECL nitrocellulose membranes. After being blocked with 5% nonfat dry milk in PBS, the membranes were incubated with the primary antibodies either 1 hour at room temperature or overnight at 4 degrees and washed with PBS-0.1% Tween 20. The membranes were finally incubated with horseradish peroxidase-linked secondary antibodies and visualized with the ECL western blot analysis system. If needed, the same membrane was stripped with Restore Western blot stripping buffer and reblotted according to the protocol provided by Pierce.

Primary antibodies for western blotting are the following: anti-Pacsin 2⁽¹⁾ 1:15,000 dilution; anti-GAPDH (Santa Cruz Biotechnology, Inc. FL-335 sc-25778) 1:1000 dilution; anti-β-actin (Sigma-Aldrich Co. LLC. A2228) 1:50000 dilution; anti-p44/42 MAPK (Erk1/2) Antibody (Cell Signaling Technology, Inc. #9102) 1:1000 dilution. Secondary antibodies (Amersham Pharmacia Biotech) were used at 1:5000 dilution.

### Immunostaining and Immunofluorescence Microscopy

Cultured cells were fixed with 4% paraformaldehyde/3% sucrose and permeabilized with 0.3% Triton X-100. Permeabilized cells were blocked with either 5% BSA or 10% goat serum for 1 hour at room temperature; and then incubated with the primary antibodies for 1 h at room temperature or overnight at 4° C. After completely washing with ice cold PBS, and incubated with a labeled secondary antibody for 1 h at room temperature. Slides were mounted with ProLong® Gold antifade reagent with DAPI (Invitrogen, Catalog # P36935). A Nikon fluorescence microscope and the SPOT camera system were used for image analysis. (Nikon, Tokyo, Japan).

Paraffin-embedded sections (4 mm), derived from perfused kidneys, were dewaxed, rehydrated through graded alcohols and boiled for 30 minutes in 10 mM citrate (pH 6.0) (Vector laboratories, CA, USA). After cooling, the sections were blocked with 5% BSA or 10% goat serum for 1 hour at room temperature and processed as cultured cells as mentioned above.

Primary antibodies for immunostaining are the following: Pacsin 2⁽¹⁾ 1:4,000 Catalog # T6793) 1/40,000 dilution, Fluorescent labeled second antibodies were all form Invitrogen and used at 1:500 dilution. Fluorescein DBA (Vector laboratories, Catalog # FL-1031) at 1/500 dilution, or fluorescein LTA (Vector Laboratories, Catalog #, FL-1321) at 1/500 dilution was used to detect respectively collecting tubules/ducts or proximal tubules.

### Renal Ischemia-Reperfusion Injury (IRI)

The IRI surgery was performed as previously described⁽²¹⁾. Briefly, animals were anesthetized with pentobarbital sodium (60 mg/kg body weight, intraperitoneally) prior to surgery. Body temperatures were controlled at 36.5-37.5° C. throughout the procedure. Kidneys were exposed through flank incisions and mice were subjected to ischemia by clamping the left renal pedicle with nontraumatic microaneurysm clamps (Roboz, Rockville, Md., USA), which were removed after 25 min (males) or 35 min (females). The right kidneys were severed as contralateral non-ischemic (control) kidneys. One milliliter of 0.9% NaCl was administered subcutaneously 2 h after surgery.

### Tubulogenesis Analysis

mIMCD3 cells were cultured in collagen I 3D gels. The collagen mixture was made with 8 volumes of type I collagen stock solution (BD Bioscience), 1 volume of concentrated DMEM (Sigma) and 1 volume of 200 mM HEPES (sigma). mIMCD3 cells (4×10³) were suspended in the collagen mixture (1 ml), and 0.85 ml of the mixture was transferred gently into 6-well tissue culture plates. After incubating the cultures at 37° C. until the collagen solidified, 2 ml of growth medium with or without 40 ng/ml HGF was added (Sigma Chemical Co., St. Louis, Mo.). Media was changed each 2-3 days. Cells were observed by phase-contrast microscopy and photographed.

### Invasion Analysis

Invasion of mIMCD3 cells into type I collagen gels was analyzed as described previously^{(11),(30)}. Briefly, transwell filters (8-μm pore size, 6.5-mm diameter; Corning, Corning, NY) were coated with type I collagen by following the manufacturer's instruction. Cells (5×10⁴) were seeded to the upper chambers without HGF, and the chambers were placed in 24-well tissue culture plates containing medium with HGF (20 ng/ml). After incubation at 37° C. for 16 hours, cultures were washed with PBS, fixed with ice-cold methanol, and stained with Giemsa staining solution. The bottom surface of the filter was filters.

### MTT Cell Proliferation and FACS Cell Cycle Analysis

To determine the cell proliferation rate of the Pacsin 2 knockdown and control mIMCD3 cells, 5,000 cells were seated in each well of a 24-well tissue culture plate. This was done in triplicate. The cells were stained with 3-[4,5-dimethylthiazol-2-yl]-2,5-diphenyl-tetrazolium bromide (MTT) at 0, 24, 48, and 72 hours; and the absorption at 590 nm was determined with a microtiter plate reader (Molecular Devices Corporation, CA). For cell cycle profile analysis, cells were synchronized at G0 phase by serum starvation for 24 hr, then allowed to reenter the cell cycle by supplying 10% serum for an additional 24 hr, and stained with propidium iodide (PI). Cells were scored by FACScan flow cytometer (Becton Dickinson). Cell cycle distribution was analyzed with Cellquest software.

### Pacsin 2 Expression and Localization in Kidney Epithelial Cells

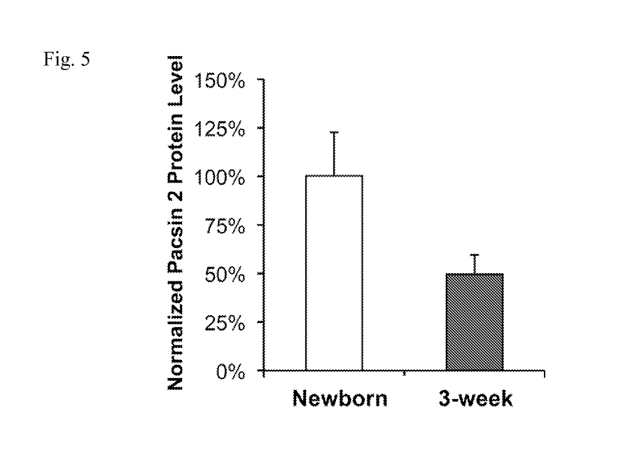

Virtually nothing is known about Pacsin 2 in the kidney. As a first step to study its role in the kidney, we double stained kidney sections from newborn and 1- to 3-week old mice with Pacsin 2 antibodies and nephron segment markers. In kidneys from newborn to 1-week old mice, Pacsin 2 expression was seen at the apical membrane in both LTA⁺ proximal and DBA⁺ collecting tubules. The apical expression of Pacsin 2 in LTA and DBA positive tubules was also seen in embryonic day 15.5 and 18.5 kidneys. (Data not shown.) At 3 weeks of age, however, Pacsin 2 expression is significantly decreased in LTA⁺ proximal tubules, and its localization becomes mostly cytoplasmic with only weak signals on the apical membrane in a small fraction of (˜5%) LTA⁺ proximal tubules. In contrast, the apical membrane expression of Pacsin 2 in DBA⁺ collecting tubules is retained and became even stronger. (Strong Pacsin 2 expression in apical membrane of LTA⁺ proximal tubules in newborn mouse kidneys, which is remarkably reduced and becomes cytosolic in 3-week old mouse kidneys. Strong apical expression of Pacsin 2 in DBA⁺ distal tubules in both newborn and 3-week old kidneys. Data not shown.) Consistently, western blot confirmed a remarkable reduction of Pacsin 2 in 3-week old kidneys compared to the newborn kidneys. (Western blot revealed a remarkable reduction of Pacsin 2 in 3-week old kidneys compared to the newborn kidneys; and the Pacsin 2 protein levels were normalized to β-Actin in each kidney data not shown; and the newborn kidneys were used for the reference and set at 100%. (n=3); FIG. 5.)

Nephrogenesis continues in the first 2 weeks of postnatal life in rodents. To amined the extreme cortex of newborn mouse kidneys where the nephrogenic zone lies. We observed strong apical labeling of Pacsin 2 in ureteric bud and its derivative structures. Only weak signals were seen in newly formed nephron structures such as the S-shaped bodies, which are negative for Pacsin 2 (Confocal images shows apical Pacsin 2 expression in the tips of ureteric bud derived structures, but not in S-shape bodies or renal vesicles. Data not shown). In the cortical medullar region of postnatal kidneys, Pacsin 2 expression is detectable in the parietal epithelial cells of the Bowman's capsule but not in the glomerulus. It is noteworthy that Pacsin 2 expression becomes detectable in both the podocytes of the glomerular tufts and parietal cells in adult kidneys, although its expression in proximal tubules becomes undetectable. (In newborn mouse kidneys, Pacsin 2 is weakly expressed in the parietal epithelial cells of the Bowman's capsule and in the glomerular tufts. There is a remarkable up-regulation of Pacsin 2 in adult 3-month old kidneys. Confocal images shows the strong expression of Pacsin 2 on the plasma membrane of podocytes of the glomerular tufts, which were labeled by WT1 staining Confocal images show that Pacsin 2 is expressed in the parietal epithelial cells of the Bowman's capsule and in the plasma membrane of the podocytes in the glomerular tufts in adult 3-months old mouse kidneys. Data not shown.)

Primary cilia act as mechano- and/or chemosensors and transduce signals from the extracellular environment into the cell to regulate a number of important signaling events. Since Pacsins may localize to the centrosomes, which is a barrel-shaped microtubule based structure critical for cell division and ciliogenesis⁽¹⁸⁾, we tested whether Pacsin 2 is on the primary cilia of kidney epithelial cells. By double-labeling cells with Pacsin 2 and acetylated α-tubulin, a marker for the axoneme of the primary cilium, we found that Pacsin 2 co-localized with acetylated α-tubulin on the shaft of the primary cilia in mIMCD3 cells (Pacsin 2 localizes on the primary cilia of mIMCD3 cells as indicated by a cilium axoneme marker acetylated-α-tubulin and a basal body marker γ-tubulin. Data not shown). Its expression on primary cilia is weakly detectable in tubular epithelial cells in vivo (Pacsin 2 is weakly expressed on the primary cilia of tubular epithelial cells in a neonatal kidney. Data not shown.). Pacsin 2 ciliary localization was further confirmed by double-labeling of Pacsin 2 with γ-tubulin, a marker for the basal body (data not shown).

Pacsin 2 Knockdown does not Affect Cell Proliferation and Cell Cycle in mIMCD3 Cells

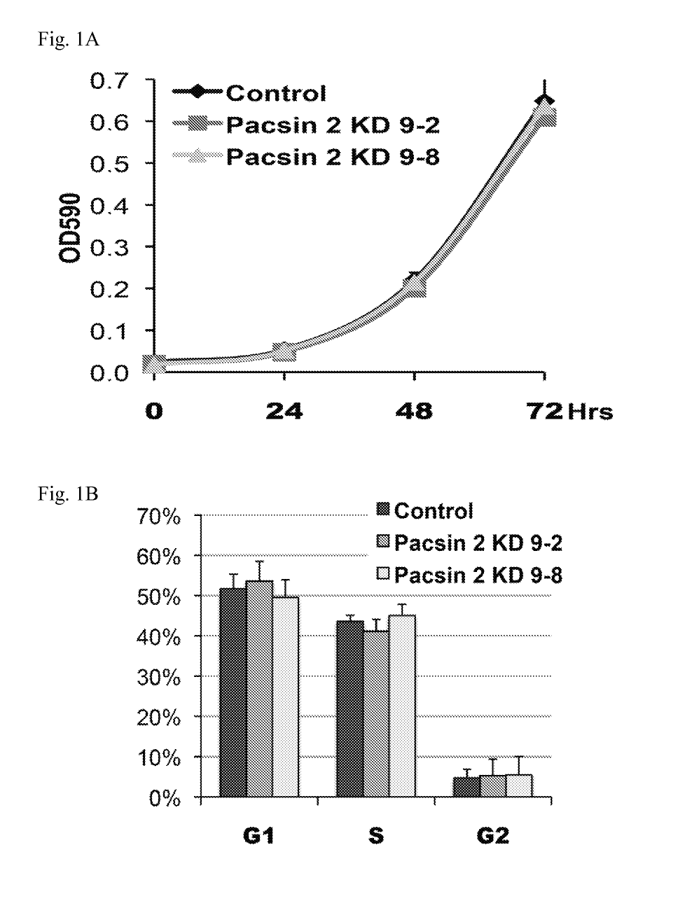

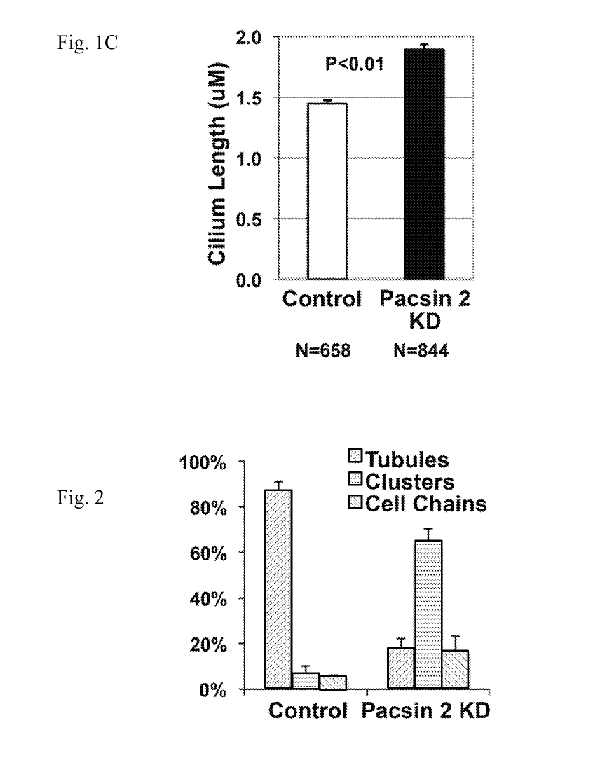

To assess the function of Pacsin 2 in kidney tubular cells, we silenced Pacsin 2 expression in mIMCD3 cells by small hairpin RNA interference (shRNA) and established four stable knockdown clones and six scrambled controls. Western blotting analysis verified a significant decrease in Pacsin 2 protein abundance in these knockdown cell lines (Data not shown). Semi-quantitative-RT-PCR confirmed that Pacsin 2 mRNA was also reduced (Significant reduction in the Pacsin 2 expression levels in 4 stable Pacsin 2 knockdown mIMCD3 clones, compared with the pSilencer control clones. Data not shown) in these four knockdown cell lines. Immunofluorescent analysis with a Pacsin 2 specific antibody revealed a significant reduction of the Pacsin 2 signal in Pacsin 2 knockdown cells, compared to control mIMCD3 cells (Secondary antibodies alone were used as a control for the specificity of the Pacsin 2 antibody. MTT assay. Data not shown.) MTT cell proliferation assay and FACS analysis revealed that neither the cell proliferation rate nor the cell cycle profile of Pacsin 2 knockdown cells was significantly altered, compared to control cells. (FIGS. 1A, 1B.)

Pacsin 2 Knockdown mIMCD3 Cells Process Longer Primary Cilia

Surprisingly, with acetylated α-tubulin as a marker, we found that the primary cilia in Pacsin 2 knockdown cells were about ˜31% longer than those in control cells. This difference is statistically significant (p<0.01), as determined by a Student's T-test (Pacsin 2 knockdown mIMCD3 cells process longer primary cilia, compared to control cells. The primary cilia axoneme was stained by acetylated α-tubulin. (Data not shown and FIG. 1C.)

### Pacsin 2 Knockdown Disrupts Tubulogenesis in 3-Dimensional Culture

mIMCD3 epithelial cells have been used as an in vitro model of the kidney tubulogenesis system. When cultured in a collagen gel matrix, mIMCD3 cells form fluid-filled branching tubule-like structures that comprise a single layer of polarized cells, resembling renal tubules. We assessed the tubulogenic potential of Pacsin 2 using this assay. After culturing in 3-dimensional (3D) type I collagen gels for 12 days, the control mIMCD3 cells formed branching tubules lined by a single layer of epithelial cells with primary cilia protruding towards the lumen (88% of the structures). This process, however, was defective in all 3 stable Pacsin 2 knockdown cell lines tested. Most of the structures formed by Pacsin 2 knockdown cells were cell clusters often containing multi-lumens (65%) or cell chains (17%). The small fraction of the Pacsin 2 knockdown cells that were able to form tubule like structures/cords showed reduced branching with no lumen formation (18%). These cords usually exhibited blunt ends, indicating that there was a branching defect. (Pacsin 2 depletion leads to defects in tubulogenesis in 3D collagen gels. Phase-contrast images for control and Pacsin 2 knockdown mIMCD3 cells cultured for 12 days in 3D type I collagen gels were taken. Quantification of structures formed in 3D tubulogenesis assays was done. The structures formed in 3D collagen gels were classified into three categories (tubule, cell cluster, and cell chain). The percentage of each type of the structures was calculated by dividing by total number of structures formed. Cell structures were counted from four individual experiments. More than 160 structures were counted for each cell type. Confocal images of cells cultured were stained with phalloidin and acetylated α-tubulin (green). Control mIMCD3 cells formed large elaborately branched tubular structures with well-established lumen lined with a single layer of epithelial cells with apical primary cilia protruding towards the lumen. The lumen for the tubule that are connected are 2 μm apart in focal plane. Pacsin 2 knockdown cells have defects in tubule formation. They tend to form tubules with blunt ends or cell clusters with isolated lumens or multi-lumens. Typical multi-lumen structures are in Pacsin 2 knockdown mIMCD3 cells. FIG. 2 and data not shown)

### Pacsin 2 Knockdown Affects Cell Invasion but not Cell Polarity

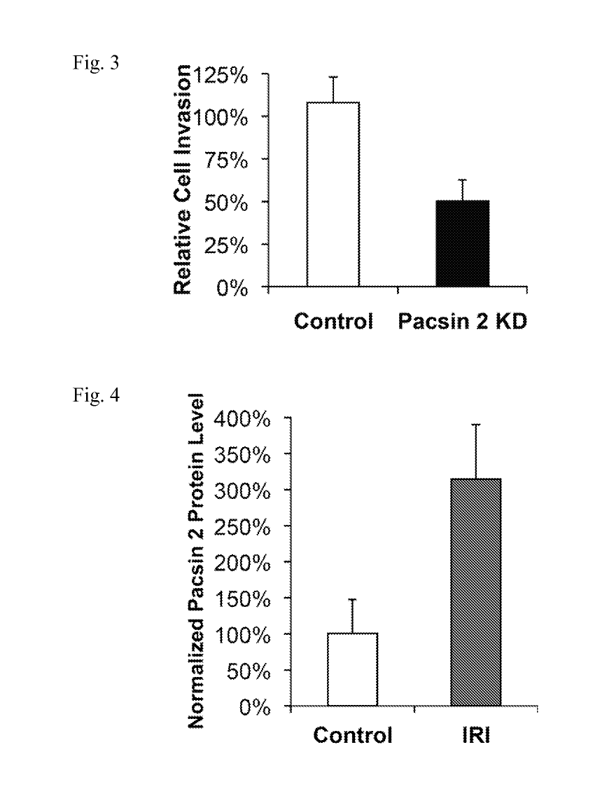

Tubulogenesis requires coordinated invasion of cells through the extracellular matrix. Pacsin family proteins interact with N-Wasp and modulate actin nucleation. Given the fact that N-Wasp deficiency in MDCK cells led to a defect in directed cell invasion, we examined whether Pacsin 2 is required for this process. Therefore, we performed an invasion assay in which subconfluent cells travel through an 8 μm porous filter which was coated with type I collagen. In this assay, an identical number of both control and Pacsin 2 knockdown cells were plated on the apical surface of the filter. The hepatic growth factor (HGF) is only added into the media of the lower chamber to attract cells to travel through the filter. Cells that traversed the filter were visualized on the opposite surface of the filter by Giemsa stain. After 16 hours of culture, there was a remarkable reduction of Pacsin 2 knockdown cells that had traveled towards the opposite side of the filter (data not shown and FIG. 3).

Epithelial tubulogenesis requires strict control of cell-cell adhesion and cell polarity^{(11, 19)}. To examine the formation of the adherent/tight junctions and cell polarity, we used ZO1 and E-cadherin as respective markers to stain cells grown on permeable filters, which allows for better maintenance of cell polarity. We did not detect obvious differences in the expression of these junctional markers between Pacsin 2 knockdown cells and control mIMCD3 cells (Pacsin 2 knockdown and control mIMCD3 cells exhibit normal tight-junctions and cell-cell adhesions as analyzed by confocal microscopy. Tight junctions are labeled with ZO1 while cell-cell adhesions are stained with E-cadherin. Pacsin 2 knockdown does not affect the apical-basal polarity. Pacsin 2 knockdown and control mIMCD3 cells exhibit normal tight junctions and cell-cell adhesions as analyzed by confocal microscopy. Tight junctions are labeled with ZO1 while cell-cell adhesions are stained with E-cadherin. Data not shown.). To determine whether there was a change in cell polarity and cell-cell junction, we stained cells cultured in 3D collagen gels with ZO1, and β-Catenin. Consistent with data from 2D cultures, both ZO1, and β-Catenin preserved a normal localization in Pacsin 2 knockdown mIMCD3 cells, compared to control mIMCD3 cells in the tubulogenesis assay (In 3D collagen gels, both ZO1 and β-Catenin preserved a normal localization in structures formed by Pacsin 2 knockdown mIMCD3 cells, compared to tubules formed by control mIMCD3 cells in a tubulogenesis assay. Data not shown.) These data suggest the preservation of apical-basal cell polarity in Pacsin 2 knockdown cells, which is consistent with the presence of primary cilia on the apical side of the multi-lumen structures in this assay (data not shown).

Pacsin 2 Expression is Upregulated after Renal Ischemia-Reperfusion Injury (IRI)

Ischemia-reperfusion injury is the most common cause of acute renal failure. It is characterized by rapid loss of renal function and tubular damages. Upon removal of the deleterious cause, the injured kidneys start a rapid repair process—the resident surviving tubular cells proliferate and migrate to replace the lost or damaged cells—24-48 hours after injury at the proximal tubules of the outer medulla where the injury is maximum^{(20, 21}). To further investigate the role of Pacsin 2 during kidney injury and the repair process, we performed unilateral renal ischemia reperfusion injury (IRI) on 3 months old wild-type mice⁽²¹⁾. Forty-eight hours after IRI, mice were sacrificed and kidneys were harvested for immunostaining. In contralateral non-ischemic kidneys that served as controls, Pacsin 2 was detected in LTA⁺ proximal tubules at basal levels in the cytoplasm. In contrast, strong Pacsin 2 labeling highlights the brush border of most LTA⁺ tubules in injured kidneys. (Pacsin 2 expression is significantly upregulated in LTA⁺ proximal tubules ischemic kidney comparing with non-ischemic kidneys, especially in the brush border. Data not shown). A more intense apical Pacsin 2 signal was also observed in DBA′ tubules in ischemic kidneys compared with that in the non-ischemic control kidneys. (Apical Pacsin 2 expression is also increased in DBA⁺ collecting tubules in ischemic kidneys. Data not shown and FIG. 4.) njured tubules with severe cell death or a denuded basement membrane (Data not shown). Consistently, western blot analysis revealed a remarkable increase in the protein levels of Pacsin 2 in injured kidneys compared to the non-ischemic control kidneys (Pacsin 2 protein levels were remarkably increased in ischemia-reperfusion injured (IRI) kidneys comparing to the non-ischemic control (CTL) kidneys. Total Erk1/2 was used as loading control in this experiment. Three pairs of kidneys were used. For each kidney, half was used for immunofluorescence analysis in and the other half for western blotting. (The protein levels of Pacsin 2 were normalized to those of total Erk1/2 in each kidney; and the non-ischemic control kidneys were used as reference and set at 100%. Data not shown.)

### REFERENCES

1. Modregger J, Ritter B, Witter B, Paulsson M, et al. All three PACSIN isoforms bind to endocytic proteins and inhibit endocytosis. Journal of Cell Science 2000; 113 Pt 24: 4511-4521.
2. Wang Q, Navarro M V, Peng G, Molinelli E, et al. Molecular mechanism of membrane constriction and tubulation mediated by the F-BAR protein Pacsin/Syndapin. Proc Natl Acad Sci USA 2009; 106: 12700-12705.
3. Plomann M, Wittmann J G, Rudolph M G. A hinge in the distal end of the PACSIN 2 F-BAR domain may contribute to membrane-curvature sensing. J Mol Biol 2010; 400: 129-136.
4. Frost A, Unger V M, De Camilli P. The BAR domain superfamily: membrane-molding macromolecules. Cell 2009; 137: 191-196.
5. Edeling M A, Sanker S, Shima T, Umasankar P K, et al. Structural requirements for PACSIN/Syndapin operation during zebrafish embryonic notochord development. PLoS One 2009; 4: e8150.
6. Qualmann B, Kelly R B. Syndapin isoforms participate in receptor-mediated endocytosis and actin organization. Journal of Cell Biology 2000; 148: 1047-1062.
7. Merrifield C J, Feldman M E, Wan L, Almers W. Imaging actin and dynamin recruitment during invagination of single clathrin-coated pits. [see comment]. Nature Cell Biology 2002; 4: 691-698.
8. Kessels M M, Qualmann B. Syndapins integrate N-WASP in receptor-mediated endocytosis. EMBO Journal 2002; 21: 6083-6094.
9. Baum B, Kunda P. Actin nucleation: spire—actin nucleator in a class of its own. Curr
10. Dharmalingam E, Haeckel A, Pinyol R, Schwintzer L, et al. F-BAR proteins of the syndapin family shape the plasma membrane and are crucial for neuromorphogenesis. J Neurosci 2009; 29: 13315-13327.
11. Yamaguchi H, Miki H, Takenawa T. Neural Wiskott-Aldrich syndrome protein is involved in hepatocyte growth factor-induced migration, invasion, and tubulogenesis of epithelial cells. Cancer Research 2002; 62: 2503-2509.
12. Merilainen J, Lehto V P, Wasenius V M. FAP52, a novel, SH3 domain-containing focal adhesion protein. Journal of Biological Chemistry 1997; 272: 23278-23284.
13. Plomann M, Lange R, Vopper G, Cremer H, et al. PACSIN, a brain protein that is upregulated upon differentiation into neuronal cells. European Journal of Biochemistry 1998; 256: 201-211.
14. Qualmann B, Roos J, DiGregorio P J, Kelly R B. Syndapin I, a synaptic dynamin-binding protein that associates with the neural Wiskott-Aldrich syndrome protein. Molecular Biology of the Cell 1999; 10: 501-513.
15. Ritter B, Modregger J, Paulsson M, Plomann M. PACSIN 2, a novel member of the PACSIN family of cytoplasmic adapter proteins. FEBS Letters 1999; 454: 356-362.
16. Kessels M M, Qualmann B. The syndapin protein family linking membrane trafficking with the cytoskeleton. Journal of Cell Science 2004; 117: 3077-3086.
17. Badano J L, Mitsuma N, Beales P L, Katsanis N. The ciliopathies: an emerging class of human genetic disorders. Annu Rev Genomics Hum Genet 2006; 7: 125-148.
18. Grimm-Gunter E M, Milbrandt M, Merkl B, Paulsson M, et al. PACSIN proteins bind tubulin and promote microtubule assembly. Experimental Cell Research 2008; 314: 1991-2003.
19. Pollack A L, Runyan R B, Mostov K E. Morphogenetic mechanisms of epithelial tubulogenesis: MDCK cell polarity is transiently rearranged without loss of cell-cell contact during scatter factor/hepatocyte growth factor-induced tubulogenesis. Developmental Biology 1998; 204: 64-79.
20. Duffield J S, Bonventre J V. Kidney tubular epithelium is restored without replacement with bone marrow-derived cells during repair after ischemic injury. Kidney Int 2005; 68: 1956-1961.
21. Takakura A, Contrino L, Zhou X, Bonventre J V, et al. Renal injury is a third hit promoting rapid development of adult polycystic kidney disease. Hum Mol Genet 2009; 18: 2523-2531.
22. Grobstein C. Trans-filter induction of tubules in mouse metanephrogenic mesenchyme. Exp Cell Res 1956; 10: 424-440.
23. Erickson R A. Inductive interactions in the development of the mouse metanephros. J Exp Zool 1968; 169: 33-42.
24. Mori K, Yang J, Barasch J. Ureteric bud controls multiple steps in the conversion of mesenchyme to epithelia. Semin Cell Dev Biol 2003; 14: 209-216.
25. Michael L, Sweeney D E, Davies J A. A role for microfilament-based contraction in branching morphogenesis of the ureteric bud. Kidney Int 2005; 68: 2010-2018.
26. de Kreuk B J, Nethe M, Fernandez-Borja M, Anthony E C, et al. The F-BAR domain protein PACSIN2 associates with Rac1 and regulates cell spreading and migration. J Cell Sci 2011; 124: 2375-2388.
27. Meng H, Tian L, Zhou J, Li Z, et al. PACSIN 2 represses cellular migration through direct association with cyclin D1 but not its alternate splice form cyclin D1b. Cell Cycle 2011; 10: 73-81.
28. Kim J, Lee J E, Heynen-Genel S, Suyama E, et al. Functional genomic screen for modulators of ciliogenesis and cilium length. Nature 2010; 464: 1048-1051.
29. Guo P, Weinstein A M, Weinbaum S. A hydrodynamic mechanosensory hypothesis for brush border microvilli. Am J Physiol Renal Physiol 2000; 279: F698-712.
30. Kong T, Xu D, Yu W, Takakura A, et al. G alpha 12 inhibits alpha2 beta1 integrin-mediated Madin-Darby canine kidney cell attachment and migration on collagen-I and blocks tubulogenesis. Mol Biol Cell 2009; 20: 4596-4610.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be d and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method of diagnosing kidney injury or a kidney disease, comprising:
assaying a biological sample obtained from a subject to determine a Pacsin 2 expression level, wherein determining the Pacsin 2 expression level is by application of a reagent that reacts with Pacsin 2 or a complex comprising Pacsin 2, and wherein the reagent comprises a detectably labeled probe;
comparing the Pacsin 2 expression level to a reference level; and
diagnosing the kidney injury or a kidney disease if the Pacsin 2 expression level is higher than the reference level.

2.-25. (canceled)

26. The method of claim 1, wherein the reagent is an antibody.

27. The method of claim 1, further comprising administering a kidney therapy if the kidney injury or kidney disease is diagnosed.

28. The method of claim 1, wherein the biological sample comprises blood or urine.

29. The method of claim 1, wherein the biological sample comprises a kidney cell, kidney tissue, a brush border of a tubule of a kidney, an apical brush border of a proximal tubule of a kidney, or combinations thereof.

30. The method of claim 1, wherein the kidney injury or kidney disease is post ischemia-reperfusion injury.

31. The method of claim 1, wherein the kidney disease is polycystic kidney disease.

32. The method of claim 27, wherein the kidney therapy is selected from the group consisting of a Pacsin 2 agonist, a Pacsin 2 product, a Pacsin 2 product mimetic and combinations thereof.

33. The method of claim 32, wherein the Pacsin 2 product comprises a Pacsin 2 protein or an active fragment thereof.

34. A method of diagnosing kidney injury or a kidney disease, comprising:
assaying a biological sample obtained from a subject to determine a Pacsin 2 expression level, wherein determining the Pacsin 2 expression level is by application of a detectably labeled probe that hybridizes to a nucleic acid molecule expressed by PACSIN2 gene;
comparing the Pacsin 2 expression level to a reference level; and
diagnosing the kidney injury or a kidney disease if the Pacsin 2 expression level is higher than the reference level.

35. The method of claim 34, wherein the nucleic acid molecule is RNA.

36. The method of claim 34, further comprising amplifying the nucleic acid molecule.

37. The method of claim 34, wherein the nucleic acid molecule or an amplification product thereof is quantified using semi-quantitative RT-PCR.

38. The method of claim 34, further comprising administering a kidney therapy if the kidney injury or kidney disease is diagnosed.

39. The method of claim 34, wherein the biological sample comprises blood or urine.

40. The method of claim 34, wherein the biological sample comprises a kidney cell, kidney tissue, a brush border of a tubule of a kidney, an apical brush border of a proximal tubule of a kidney, or combinations thereof.

41. The method of claim 34, wherein the kidney injury or kidney disease is post ischemia-reperfusion injury.

42. The method of claim 34, wherein the kidney disease is polycystic kidney disease.

43. The method of claim 38, wherein the kidney therapy is selected from the group consisting of a Pacsin 2 agonist, a Pacsin 2 product, a Pacsin 2 product mimetic and combinations thereof.

44. The method of claim 43, wherein the Pacsin 2 product comprises a Pacsin 2 protein or an active fragment thereof.


SEQUENCE LISTING (2 sequences)

SEQ ID NO: 1 - protein, 486 aa, Homo sapiens
MSVTYDDSVGVEVSSDSFWEVGNYKRTVKRIDDGHRLCSDLMNCLHERARIEKAYAQQLTEWARRWRQLVEKGPQYGTVEKAWMAFMSEAERVSELHLEVKASLMNDDFEKIKNWQKEAFHKQMMGGFKETKEAEDGFRKAQKPWAKKLKEVEAAKKAHHAACKEEKLAISREANSKADPSLNPEQLKKLQDKIEKCKQDVLKTKEKYEKSLKELDQGTPQYMENMEQVFEQCQQFEEKRLRFFREVLLEVQKHLDLSNVAGYKAIYHDLEQSIRAADAVEDLRWFRANHGPGMAMNWPQFEEWSADLNRTLSRREKKKATDGVTLTGINQTGDQSLPSKPSSTLNVPSNPAQSAQSQSSYNPFEDEDDTGSTVSEKDDTKAKNVSSYEKTQSYPTDWSDDESNNPFSSTDANGDSNPFDDDATSGTEVRVRALYDYEGQEHDELSFKAGDELTKMEDEDEQGWCKGRLDNGQVGLYPANYVEAIQ

SEQ ID NO: 2 - DNA, 19 bp, Murine
atgtctgtcacctacgatg